(19) United States
(12) Patent Application Publication
(10) Pub. No.: US 2024/0130994 A1
(43) Pub. Date: Apr. 25, 2024

(54) IONIC LIQUID FORMULATIONS FOR TREATING DIABETES

(71) Applicant: i2O Therapeutics, Inc., Cambridge, MA (US)

(72) Inventors: Tyler Brown, Cambridge, MA (US); Kelly IBSEN, Boston, MA (US)

(21) Appl. No.: 18/176,094

(22) Filed: Feb. 28, 2023

Related U.S. Application Data
(63) Continuation of application No. PCT/US2021/048537, filed on Aug. 31, 2021.
(60) Provisional application No. 63/073,172, filed on Sep. 1, 2020.
(60) Provisional application No. 63/154,461, filed on Feb. 26, 2021.
(60) Provisional application No. 63/160,575, filed on Mar. 12, 2021.

(51) Int. Cl. A61K 31/205; A61K 31/185; A61K 31/191; A61K 31/192; A61K 31/194; A61K 31/195; A61K 31/198; A61K 31/20; A61K 31/201; A61K 31/202; A61K 31/222; A61K 31/352; A61K 31/375; A61K 31/385; A61K 31/4015; A61K 31/575; A61K 33/42; A61K 38/17; A61P 3/10

(52) U.S. Cl. CPC A61K 31/205; A61K 31/185; A61K 31/191; A61K 31/192; A61K 31/194; A61K 31/195; A61K 31/198; A61K 31/20; A61K 31/201; A61K 31/202; A61K 31/222; A61K 31/352; A61K 31/375; A61K 31/385; A61K 31/4015; A61K 31/575; A61K 33/42; A61K 38/1796; A61P 3/10

(57) ABSTRACT

Disclosed herein are ionic liquids and deep eutectic liquids for the treatment of diabetes and related diseases including obesity and metabolic disorders.

### RELATED APPLICATIONS

This application is a continuation of International Application No. PCT/US2021/048537, filed on Aug. 31, 2021, which claims the benefit of U.S. Provisional Patent Application No. 63/073,172 filed on Sep. 1, 2020, U.S. Provisional Patent Application No. 63/154,461 filed on Feb. 26, 2021, and U.S. Provisional Patent Application No. 63/160,575 filed on Mar. 12, 2021, the entire contents of each of which are hereby incorporated by reference.

### REFERENCE TO A SEQUENCE LISTING XML

This application contains a Sequence Listing which has been submitted electronically in XML format. The Sequence Listing XML is incorporated herein by reference. Said XML file, created on Apr. 10, 2023, is named 56017-707_301_SL.xml and is 6,219 bytes in size.

### BACKGROUND OF THE INVENTION

Diabetes is a metabolic disease characterized by the inability of the pancreas to secrete a level of insulin adequate to maintain a normal level of systemic glucose. Despite advances, there remains a need for novel treatments of diabetes.

### SUMMARY OF THE INVENTION

Provided herein, in one aspect, is a method of treating a disease or disorder in a subject in need thereof, comprising administering to the subject a therapeutically effective amount of a composition comprising an ionic liquid.

In some embodiments, the disease or disorder is diabetes. In some embodiments, the disease or disorder is Type 1 diabetes. In some embodiments, the disease or disorder is Type 2 diabetes. In some embodiments, the disease or disorder is non-alcoholic steatohepatitis.

Provided herein, in another aspect, is a method of treating obesity, preventing weight gain, or reducing weight in a subject in need thereof, comprising administering to the subject a therapeutically effective amount of a composition comprising an ionic liquid.

In some embodiments, the composition is administered via subcutaneous, intravenous, or oral administration. In some embodiments, the composition is administered via oral administration. In some embodiments, the composition is administered as a liquid-filled capsule. In some embodiments, the composition is administered in a single dose. In some embodiments, the composition is administered in multiple doses. In some embodiments, the composition is administered to a mucus membrane.

In some embodiments, the composition comprises the ionic liquid at a concentration of at least 0.1% weight per volume. In some embodiments, the composition comprises the ionic liquid at a concentration of at least 0.05 M. In some embodiments, the ionic liquid comprises a cation:anion ratio of from about 4:1 to about 1:4.

In some embodiments, the ionic liquid is represented by Formula (I):

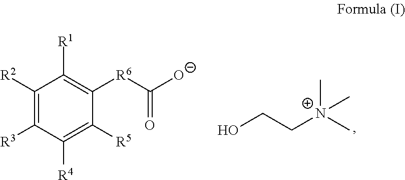

wherein:

R¹, R², R³, R⁴, and R⁵ are independently selected from hydrogen, halo, cyano, nitro, amino, C₁₋₆alkoxy, C₁₋6heteroalkyl, C₁₋6haloalkyl, C₁₋₆alkyl, C₂₋₆alkenyl, and C₂₋₆alkynyl; and

R⁶ is selected from C₁₋₆alkyl, C₂₋₆alkenyl, and C₂₋₆alkynyl.

In some embodiments, at least two of R¹, R², R³, R⁴, and R⁵ are hydrogen. In some embodiments, at least three of R¹, R², R³, R⁴, and R⁵ are hydrogen. In some embodiments, R¹, R², R³, R⁴, and R⁵ are hydrogen.

In some embodiments, R⁶ is selected from C₁₋₆alkyl and C₂₋₆alkenyl. In some embodiments, R⁶ is C₁₋₆alkyl. In some embodiments, R⁶ is C₂alkyl. In some embodiments, R⁶ is C₁₋₆alkenyl. In some embodiments, R⁶ is C₂alkenyl.

In some embodiments, the ionic liquid is represented by Formula (II):

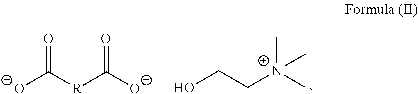

wherein:

R is selected from C₁₋₆alkyl, C₂₋₆alkenyl, and C₂₋₆alkynyl.

In some embodiments, R is C₁₋₆alkyl. In some embodiments, R is C₁alkyl. In some embodiments, R is C₃alkyl.

In some embodiments, the ionic liquid is represented by Formula (III):

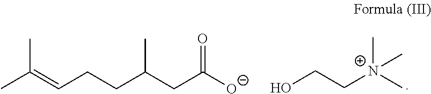

In some embodiments, the ionic liquid comprises a cholinium cation and an anion selected from cinnamate, hydrocinnamate, malonate, citronellate, and glutarate. In some embodiments, the anion is selected from cinnamate, hydrocinnamate, and citronellate.

In some embodiments, the composition further comprises one or more additional agents. In some embodiments, the one or more additional agents are selected from a nucleic acid, a small molecule, and a polypeptide. In some embodiments, the one or more additional agents comprise a nucleic acid. In some embodiments, the one or more additional agents comprise a small molecule. In some embodiments, the one or more additional agents comprise a polypeptide.

In some embodiments, the one or more additional agents are selected from a glucagon-like peptide (GLP-1), a glucagon-like peptide derivative, and a glucagon-like peptide mimetic. In some embodiments, the one or more additional agents are selected from liraglutide, exenatide, and semaglutide. In some embodiments, the one or more additional agents comprise liraglutide.

In some embodiments, the one or more additional agents are selected from insulin and pramlintide.

In some embodiments, the composition further comprises a pharmaceutically acceptable excipient.

Provided herein, in another aspect, is a composition comprising an ionic liquid, wherein the ionic liquid comprises choline and hydrocinnamic acid in a 1:2 molar ratio.

Provided herein, in another aspect, is a composition comprising an ionic liquid, wherein the ionic liquid comprises choline and cinnamic acid in a 1:1 molar ratio.

Provided herein, in another aspect, is a composition comprising an ionic liquid, wherein the ionic liquid comprises choline and glutaric acid in a 1:1 molar ratio.

Provided herein, in another aspect, is a composition comprising an ionic liquid, wherein the ionic liquid comprises choline and malonic acid in a 1:1 molar ratio.

Provided herein, in another aspect, is a composition comprising an ionic liquid, wherein the ionic liquid comprises choline and octenoic acid in a 1:1 molar ratio.

Provided herein, in another aspect, is a composition comprising an ionic liquid, wherein the ionic liquid comprises choline and octenoic acid in a 1:2 molar ratio.

Provided herein, in another aspect, is a composition comprising an ionic liquid, wherein the ionic liquid comprises choline and citronellic acid in a 1:1 molar ratio.

Provided herein, in another aspect, is a composition comprising an ionic liquid, wherein the ionic liquid comprises choline and citronellic acid in a 1:2 molar ratio.

In some embodiments, the composition further comprises one or more additional agents. In some embodiments, the one or more additional agents are selected from a nucleic acid, a small molecule, and a polypeptide. In some embodiments, the one or more additional agents comprise a nucleic acid. In some embodiments, the one or more additional agents comprise a small molecule. In some embodiments, the one or more additional agents comprise a polypeptide.

In some embodiments, the one or more additional agents are selected from a glucagon-like peptide (GLP-1), a glucagon-like peptide derivative, and a glucagon-like peptide mimetic. In some embodiments, the one or more additional agents are selected from liraglutide, exenatide, and semaglutide. In some embodiments, the one or more additional agents comprise liraglutide.

In some embodiments, the one or more additional agents are selected from insulin and pramlintide.

Provided herein, in another aspect, is a pharmaceutical composition comprising a composition described herein and a pharmaceutically acceptable excipient

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the amino acid sequence of glucagon-like peptide-1 (GLP-1) (SEQ ID NO: 1).

FIG. 2 shows the amino acid sequence of Exenatide (SEQ ID NO: 2).

FIG. 3 shows the amino acid sequence of Liraglutide (SEQ ID NO: 3).

FIG. 4 shows the amino acid sequence of Semaglutide (SEQ ID NO: 4).

FIG. 5 shows various ionic liquids which are liquid at room temperature.

FIG. 6 shows various ionic liquids which are not liquid at room temperature.

FIG. 7 shows the change in blood glucose levels over time following intrajejunal administration of choline-citronellic acid to non-diabetic rats.

FIG. 8 shows the change in blood glucose levels over time following administration of choline-octanoic acid and choline-octenoic acid to non-diabetic rats.

FIG. 9 shows the change in blood glucose levels over time following administration of citronellic acid to non-diabetic rats.

FIG. 10 shows the change in blood glucose levels over time following subcutaneous and oral administration of choline-citronellic acid to non-diabetic rats.

FIG. 11 shows the change in blood glucose levels over time following oral administration of choline-citronellic acid to diabetic rats.

FIG. 12 shows the change in plasma insulin levels over time following intrajejunal, subcutaneous, and oral administration of choline-citronellic acid to diabetic rats.

FIG. 13 shows the change in blood glucose levels and glucose levels in the urine over time following intrajejunal administration of choline-citronellic acid to non-diabetic rats.

FIG. 14 shows the change in serum liraglutide levels over time following intrajejunal administration of choline-hydrocinnamic acid and liraglutide to non-diabetic rats.

FIG. 15 shows Liraglutide delivery in C-Cinnamic acid 1:1 to the duodenum (liquid) or stomach of dogs (liquid or capsule) compared to IV (intravenous), SC (subcutaneous) dosing and oral unformulated Liraglutide to the stomach.

FIG. 16 shows Exen in C-Cinnamic acid 1:1 to stomach of dogs as a liquid compared to IV, SC dosing and unformulated (Exenatide-Saline).

FIG. 17 shows Semaglutide in Choline-Cinnamic Acid 1:1 delivered to the stomach in 0, 00 or 000 gelatin capsules coated with Evonik EPO coating or 0 HPMC capsules.

FIG. 18 shows Semaglutide delivery in C-Cinnamic acid 1:1 to the stomach of dogs (liquid and capsule) compared to IV, SC dosing and oral unformulated (PPB) or with SNAC (SNAC-PPB).

FIG. 19 shows co-delivery of Liraglutide and Exenatide with Choline-Cinnamic Acid 1:1.

FIG. 20 shows H&E-stained GI (gastrointestinal) tract tissues for Ionic Liquid (IL) and saline dosed rats at 100 μL.

FIG. 21 shows blood and plasma results for 100 μL Ionic Liquid (IL)-dosed (light grey; left columns) and saline-dosed (dark grey; right columns) rats.

FIG. 22 shows immunohistochemistry staining of jejunum tight junctions from Ionic Liquid (IL)-dose and saline-dosed (placebo) groups stained for Occuldin and Claudin-1.

FIG. 23 shows body weights of rats in 100 μL dosed Ionic Liquid (IL) and placebo group.

### DETAILED DESCRIPTION OF THE INVENTION

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene— refers to a substituted or unsubstituted alkylene chain with from x to y carbons in the alkylene chain. For example —C₁₋₆alkylene— may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

The term “ionic liquids” as used herein refers to organic salts or mixtures of organic salts which exist in a liquid state. Ionic liquids have been shown to be useful in a variety of fields, including in industrial processing, catalysis, pharmaceuticals, and electrochemistry. The ionic liquids contain at least one anionic and at least one cationic component. Ionic liquids can comprise an additional hydrogen bond donor (i.e. any molecule that can provide an —OH or an —NH group); examples include but are not limited to alcohols, fatty acids, and amines. The anionic and the cationic component may be present in any molar ratio. Exemplary molar ratios (cation:anion) include but are not limited to 1:1, 1:2, 2:1, and ranges between these ratios. In some embodiments, the ionic liquid or solvent exists as a liquid below 100° C. In some embodiments, the ionic liquid or solvent exists as a liquid at room temperature.

The phrase “pharmaceutically acceptable excipient” or “pharmaceutically acceptable carrier” as used herein means a pharmaceutically acceptable material, composition or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

As used herein, the terms “subject” and “patient” include animals (e.g., vertebrates, amphibians, fish, mammals, cats, dogs, horses, pigs, cows, sheep, rodents, rabbits, squirrels, bears, and primates (e.g., chimpanzees, gorillas, and humans)). The subject is preferably a mammal. The mammal can be, e.g., any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

A “control” or “standard control” refers to a sample, measurement, or value that serves as a reference, usually a known reference, for comparison to a test sample, measurement, or value. For example, a test sample can be taken from a subject having a given disease (e.g., diabetes) and compared with a known normal (non-diseased) individual (e.g., a standard control subject). A standard control can also represent an average measurement or value gathered from a population of similar individuals (e.g., standard control subjects) that do not have a given disease (i.e., standard control population), e.g., healthy individuals with a similar medical background, same age, weight, etc. A standard control value can also be obtained from the same individual, e.g., from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters.

As used herein, a “drug” is any agent which will exert an effect on a target cell or organism. A drug can be selected from a group comprising: chemicals; small organic or inorganic molecules; peptide; protein; or nucleic acid. Non-limiting examples of active compounds contemplated for use in the methods described herein include small molecules, polypeptides, nucleic acids, antibodies, vaccines, a GLP-1 polypeptide or mimetic/analog thereof, pramlintide, and insulin.

As used herein, “diabetes” refers to diabetes mellitus, a metabolic disease characterized by a deficiency or absence of insulin secretion by the pancreas. As used throughout, “diabetes” includes all types including Type 1 and Type 2 diabetes mellitus unless otherwise specified herein. The two most common forms of diabetes are due to either a diminished production of insulin (in Type 1), or diminished response by the body to insulin (in Type 2). In Type 1 diabetes, the function of the pancreas is progressively lost, thus eventually making the patient entirely dependent on the exogenously delivered insulin for the management of diabetes. In Type 2, the patient maintains some functioning of the pancreas, but the sensitivity of the body to insulin is reduced, thus reducing the extent of glycemia maintained by the patient. Type 2 patients are treated by a variety of drugs including oral medications that increase glucose sensitivity, GLP-1 analogs, or insulin. Both types of diabetes lead to hyperglycemia, which causes the acute signs of diabetes: excessive urine production, increased thirst and increased fluid intake, blurred vision, unexplained weight loss, lethargy, and changes in energy metabolism. Diabetes can cause many complications including neuropathy, retinopathy, poor microvascular function, renal failure, and poor wound healing. A “pre-diabetic” subject can be characterized, for example, as having elevated fasting blood sugar or elevated post-prandial blood sugar such that the glucose levels do not fit the current medical definitions of diabetes. A “newly diagnosed” subject refers to a Type 1 diabetic patient that is within 1-3 years of their diagnosis. This patient population can be physiologically or emotionally different from the general Type 1 diabetic population.

The term “obesity” refers to excess fat in the body. Obesity can be determined by any measure accepted and utilized by those of skill in the art. Currently, an accepted measure of obesity is body mass index (BMI). Consequences of obesity include cardiovascular disease, high blood pressure (i.e., hypertension), osteoarthritis, cancer, and diabetes.

### Compositions Comprising Ionic Liquids

Provided herein, in some embodiments, are compositions comprising ionic liquids useful in the treatment of certain diseases and disorders. In some embodiments, the anion in the ionic liquid may be chosen from cinnamic acid, hydrocinnamic acid, hydroxycinnamic (3-phenylpropanoic or benzylacetic) acid, methoxycinnamic acid, ferulic acid, isoferulic acid, 2-phenylpropionic (hydratropic acid), coumaric acid, 3,3-diphenylpropionic acid, 3,5-dimethoxy-4-hydroxy-cinnamic acid (sinapinic acid). Other structural analogs of cinnamic acid may be used.

In some embodiments, the ionic liquid comprises choline and hydrocinnamic acid in a 1:2 molar ratio. In some embodiments, the ionic liquid comprises choline and cinnamic acid in a 1:1 molar ratio. In some embodiments, the ionic liquid comprises choline and glutaric acid in a 1:1 molar ratio. In some embodiments, the ionic liquid comprises choline and malonic acid in a 1:1 molar ratio. In some embodiments, the ionic liquid comprises choline and octenoic acid in a 1:1 molar ratio. In some embodiments, the ionic liquid comprises choline and octenoic acid in a 1:2 molar ratio. In some embodiments, the ionic liquid comprises choline and citronellic acid in a 1:1 molar ratio. In some embodiments, the ionic liquid comprises choline and citronellic acid in a 1:2 molar ratio.

In some embodiments, a structural analog of cinnamic acid is represented by the formula:

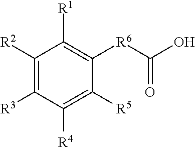

wherein:

R¹, R², R³, R⁴, and R⁵ are independently selected from hydrogen, halo, cyano, nitro, amino, C₁₋₆alkoxy, C₁₋6heteroalkyl, C₁₋6haloalkyl, C₁₋₆alkyl, C₂₋₆alkenyl, and C₂₋₆alkynyl; and

R⁶ is selected from C₁₋₆alkyl, C₂₋₆alkenyl, and C₂₋₆alkynyl.

In some embodiments, the anion is a diacid represented by the formula:

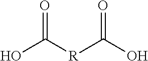

wherein:

R is selected from C₁₋₆alkyl, C₂₋₆alkenyl, and C₂₋₆alkynyl.

Choline (or cholinium) is a suitable choice of cation in the preparation of ionic liquids. However, the cation can be chosen from a variety of molecules including salts of choline (e.g., choline chloride), derivates of choline, or any other biocompatible cation that is able to form an ionic liquid to with the anions described herein.

In some embodiments, the ionic liquid is prepared by mixing an acid with choline bicarbonate, as exemplified by the scheme below:

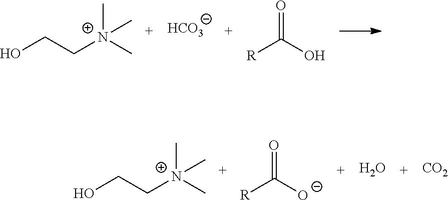

Choline bicarbonate reacts with a carboxylic acid to form water, carbon dioxide, and an ionic liquid represented by the formula:

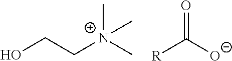

Depending on the ratio of anion and cation in the reaction mixture, the resultant mixture may also contain either excess acid or excess choline bicarbonate. The term “ionic liquid” used herein includes all stoichiometries, including equimolar acid and choline carbonate, excess acid, or excess choline bicarbonate.

The ionic liquid structures shown with or without an acidic proton are equivalent and interchangeable depending on the concentration and composition.

In some embodiments, the properties of an ionic liquid are determined by the ionic interactions between the anion and the cation. In some embodiments, the properties of an ionic liquid are determined by the hydrogen bonding interactions between the anion and cation. The relative contribution of ionic and hydrogen bonding interactions to the properties of the ionic liquid may vary depending on the nature of the ions.

In some embodiments, the composition comprises the ionic liquid at a concentration of at least 0.01% weight per volume. In some embodiments, the composition comprises the ionic liquid at a concentration of at least 0.02% weight per volume. In some embodiments, the composition comprises the ionic liquid at a concentration of at least 0.03% weight per volume. In some embodiments, the composition comprises the ionic liquid at a concentration of at least 0.04% weight per volume. In some embodiments, the composition comprises the ionic liquid at a concentration of at least 0.05% weight per volume. In some embodiments, the composition comprises the ionic liquid at a concentration of at least 0.06% weight per volume. In some embodiments, the composition comprises the ionic liquid at a concentration of at least 0.07% weight per volume. In some embodiments, the composition comprises the ionic liquid at a concentration of at least 0.08% weight per volume. In some embodiments, the composition comprises the ionic liquid at a concentration of at least 0.09% weight per volume. In some embodiments, the composition comprises the ionic liquid at a concentration of at least 0.1% weight per volume. In some embodiments, the composition comprises the ionic liquid at a concentration of at least 0.2% weight per volume. In some embodiments, the composition comprises the ionic liquid at a concentration of at least 0.3% weight per volume. In some embodiments, the composition comprises the ionic liquid at a concentration of at least 0.4% weight per volume. In some embodiments, the composition comprises the ionic liquid at a concentration of at least 0.5% weight per volume. In some embodiments, the composition comprises the ionic liquid at a concentration of at least 0.6% weight per volume. In some embodiments, the composition comprises the ionic liquid at a concentration of at least 0.7% weight per volume. In some embodiments, the composition comprises the ionic liquid at a concentration of at least 0.8% weight per volume. In some embodiments, the composition comprises the ionic liquid at a concentration of at least 0.9% weight per volume. In some embodiments, the composition comprises the ionic liquid at a concentration of at least 1.0% weight per volume.

In some embodiments, the composition comprises the ionic liquid at a concentration of at least 0.01 M. In some embodiments, the composition comprises the ionic liquid at a concentration of at least 0.02 M. In some embodiments, the composition comprises the ionic liquid at a concentration of at least 0.03 M. In some embodiments, the composition comprises the ionic liquid at a concentration of at least 0.04 M. In some embodiments, the composition comprises the ionic liquid at a concentration of at least 0.05 M. In some embodiments, the composition comprises the ionic liquid at a concentration of at least 0.06 M. In some embodiments, the composition comprises the ionic liquid at a concentration of at least 0.07 M. In some embodiments, the composition comprises the ionic liquid at a concentration of at least 0.08 M. In some embodiments, the composition comprises the ionic liquid at a concentration of at least 0.09 M. In some embodiments, the composition comprises the ionic liquid at a concentration of at least 0.1 M. In some embodiments, the composition comprises the ionic liquid at a concentration of at least 0.2 M. In some embodiments, the composition comprises the ionic liquid at a concentration of at least 0.3 M. In some embodiments, the composition comprises the ionic liquid at a concentration of at least 0.4 M. In some embodiments, the composition comprises the ionic liquid at a concentration of at least 0.5 M. In some embodiments, the composition comprises the ionic liquid at a concentration of at least 0.6 M. In some embodiments, the composition comprises the ionic liquid at a concentration of at least 0.7 M. In some embodiments, the composition comprises the ionic liquid at a concentration of at least 0.8 M. In some embodiments, the composition comprises the ionic liquid at a concentration of at least 0.9 M. In some embodiments, the composition comprises the ionic liquid at a concentration of at least 1.0 M.

In some embodiments, the ionic liquid comprises a cation:anion ratio of from about 4:1 to about 1:4. In some embodiments, the ionic liquid comprises a cation:anion ratio of about 4.4:1. In some embodiments, the ionic liquid comprises a cation:anion ratio of about 4.3:1. In some embodiments, the ionic liquid comprises a cation:anion ratio of about 4.2:1. In some embodiments, the ionic liquid comprises a cation:anion ratio of about 4.1:1. In some embodiments, the ionic liquid comprises a cation:anion ratio of about 4.0:1. In some embodiments, the ionic liquid comprises a cation:anion ratio of about 3.9:1. In some embodiments, the ionic liquid comprises a cation:anion ratio of about 3.8:1. In some embodiments, the ionic liquid comprises a cation:anion ratio of about 3.7:1. In some embodiments, the ionic liquid comprises a cation:anion ratio of about 3.6:1. In some embodiments, the ionic liquid comprises a cation:anion ratio of about 3.5:1. In some embodiments, the ionic liquid comprises a cation:anion ratio of about 3.4:1. In some embodiments, the ionic liquid comprises a cation:anion ratio of about 3.3:1. In some embodiments, the ionic liquid comprises a cation:anion ratio of about 3.2:1. In some embodiments, the ionic liquid comprises a cation:anion ratio of about 3.1:1. In some embodiments, the ionic liquid comprises a cation:anion ratio of about 3.0:1. In some embodiments, the ionic liquid comprises a cation:anion ratio of about 2.9:1. In some embodiments, the ionic liquid comprises a cation:anion ratio of about 2.8:1. In some embodiments, the ionic liquid comprises a cation:anion ratio of about 2.7:1. In some embodiments, the ionic liquid comprises a cation:anion ratio of about 2.6:1. In some embodiments, the ionic liquid comprises a cation:anion ratio of about 2.5:1. In some embodiments, the ionic liquid comprises a cation:anion ratio of about 2.4:1. In some embodiments, the ionic liquid comprises a cation:anion ratio of about 2.3:1. In some embodiments, the ionic liquid comprises a cation:anion ratio of about 2.2:1. In some embodiments, the ionic liquid comprises a cation:anion ratio of about 2.1:1. In some embodiments, the ionic liquid comprises a cation:anion ratio of about 2:1. In some embodiments, the ionic liquid comprises a cation:anion ratio of about 1.9:1. In some embodiments, the ionic liquid comprises a cation:anion ratio of about 1.8:1. In some embodiments, the ionic liquid comprises a cation:anion ratio of about 1.7:1. In some embodiments, the ionic liquid comprises a cation:anion ratio of about 1.6:1. In some embodiments, the ionic liquid comprises a cation:anion ratio of about 1.5:1. In some embodiments, the ionic liquid comprises a cation:anion ratio of about 1.4:1. In some embodiments, the ionic liquid comprises a cation:anion ratio of about 1.3:1. In some embodiments, the ionic liquid comprises a cation:anion ratio of about 1.2:1. In some embodiments, the ionic liquid comprises a cation:anion ratio of about 1.1:1. In some embodiments, the ionic liquid comprises a cation:anion ratio of about 1:1. In some embodiments, the ionic liquid comprises a cation:anion ratio of about 1:1.1. In some embodiments, the ionic liquid comprises a cation:anion ratio of about 1:1.2. In some embodiments, the ionic liquid comprises a cation:anion ratio of about 1:1.3. In some embodiments, the ionic liquid comprises a cation:anion ratio of about 1:1.4. In some embodiments, the ionic liquid comprises a cation:anion ratio of about 1:1.5. In some embodiments, the ionic liquid comprises a cation:anion ratio of about 1:1.6. In some embodiments, the ionic liquid comprises a cation:anion ratio of about 1:1.7. In some embodiments, the ionic liquid comprises a cation:anion ratio of about 1:1.8. In some embodiments, the ionic liquid comprises a cation:anion ratio of about 1:1.9. In some embodiments, the ionic liquid comprises a cation:anion ratio of about 1:2. In some embodiments, the ionic liquid comprises a cation:anion ratio of about 1:2.1. In some embodiments, the ionic liquid comprises a cation:anion ratio of about 1:2.2. In some embodiments, the ionic liquid comprises a cation:anion ratio of about 1:2.3. In some embodiments, the ionic liquid comprises a cation:anion ratio of about 1:2.4. In some embodiments, the ionic liquid comprises a cation:anion ratio of about 1:2.5. In some embodiments, the ionic liquid comprises a cation:anion ratio of about 1:2.6. In some embodiments, the ionic liquid comprises a cation:anion ratio of about 1:2.7. In some embodiments, the ionic liquid comprises a cation:anion ratio of about 1:2.8. In some embodiments, the ionic liquid comprises a cation:anion ratio of about 1:2.9. In some embodiments, the ionic liquid comprises a cation:anion ratio of about 1:3.0. In some embodiments, the ionic liquid comprises a cation:anion ratio of about 1:3.1. In some embodiments, the ionic liquid comprises a cation:anion ratio of about 1:3.2. In some embodiments, the ionic liquid comprises a cation:anion ratio of about 1:3.3. In some embodiments, the ionic liquid comprises a cation:anion ratio of about 1:3.4. In some embodiments, the ionic liquid comprises a cation:anion ratio of about 1:3.5. In some embodiments, the ionic liquid comprises a cation:anion ratio of about 1:3.6. In some embodiments, the ionic liquid comprises a cation:anion ratio of about 1:3.7. In some embodiments, the ionic liquid comprises a cation:anion ratio of about 1:3.8. In some embodiments, the ionic liquid comprises a cation:anion ratio of about 1:3.9. In some embodiments, the ionic liquid comprises a cation:anion ratio of about 1:4.0. In some embodiments, the ionic liquid comprises a cation:anion ratio of about 1:4.1. In some embodiments, the ionic liquid comprises a cation:anion ratio of about 1:4.2. In some embodiments, the ionic liquid comprises a cation:anion ratio of about 1:4.3. In some embodiments, the ionic liquid comprises a cation:anion ratio of about 1:4.4.

In some embodiments, the composition comprises the ionic liquid at a concentration of at least 0.1% weight per volume. In some embodiments, the composition comprises the ionic liquid at a concentration of at least 0.05 M.

In some embodiments, the ionic liquid comprises a cation:anion ratio of from about 2:1 to about 1:2. In some embodiments, the ionic liquid comprises a cation:anion ratio of about 2.2:1. In some embodiments, the ionic liquid comprises a cation:anion ratio of about 2.1:1. In some embodiments, the ionic liquid comprises a cation:anion ratio of about 2:1. In some embodiments, the ionic liquid comprises a cation:anion ratio of about 1.9:1. In some embodiments, the ionic liquid comprises a cation:anion ratio of about 1.8:1. In some embodiments, the ionic liquid comprises a cation:anion ratio of about 1.7:1. In some embodiments, the ionic liquid comprises a cation:anion ratio of about 1.6:1. In some embodiments, the ionic liquid comprises a cation:anion ratio of about 1.5:1. In some embodiments, the ionic liquid comprises a cation:anion ratio of about 1.4:1. In some embodiments, the ionic liquid comprises a cation:anion ratio of about 1.3:1. In some embodiments, the ionic liquid comprises a cation:anion ratio of about 1.2:1. In some embodiments, the ionic liquid comprises a cation:anion ratio of about 1.1:1. In some embodiments, the ionic liquid comprises a cation:anion ratio of about 1:1. In some embodiments, the ionic liquid comprises a cation:anion ratio of about 1:1.1. In some embodiments, the ionic liquid comprises a cation:anion ratio of about 1:1.2. In some embodiments, the ionic liquid comprises a cation:anion ratio of about 1:1.3. In some embodiments, the ionic liquid comprises a cation:anion ratio of about 1:1.4. In some embodiments, the ionic liquid comprises a cation:anion ratio of about 1:1.5. In some embodiments, the ionic liquid comprises a cation:anion ratio of about 1:1.6. In some embodiments, the ionic liquid comprises a cation:anion ratio of about 1:1.7. In some embodiments, the ionic liquid comprises a cation:anion ratio of about 1:1.8. In some embodiments, the ionic liquid comprises a cation:anion ratio of about 1:1.9. In some embodiments, the ionic liquid comprises a cation:anion ratio of about 1:2. In some embodiments, the ionic liquid comprises a cation:anion ratio of about 1:2.1. In some embodiments, the ionic liquid comprises a cation:anion ratio of about 1:2.2. In some embodiments, the ionic liquid comprises a cation:anion ratio of about 1:2.3. In some embodiments, the ionic liquid comprises a cation:anion ratio of about 1:2.4. In some embodiments, the ionic liquid comprises a cation:anion ratio of about 1:2.5.

In some embodiments, the ionic liquid comprises any one of the cations listed in Table 1. In some embodiments, the ionic liquid comprises any one or more of the cations listed in Table 1. In some embodiments, the ionic liquid comprises any one of the anions listed in Table 1. In some embodiments, the ionic liquid comprises any one or more of the anions listed in Table 1. In some embodiments, the ionic liquid comprises any one of the cation:anion ratio listed in Table 1. In some embodiments, the composition as provided herein comprises any one of the ionic liquids listed in Table 1. In some embodiments, the composition as provided herein comprises any one or more of the ionic liquids listed in Table 1. In some embodiments, the composition as provided herein comprises any one of the ionic liquids in any one of the cation:anion ratios listed in Table 1. In some embodiments, the composition as provided herein comprises any one or more of the ionic liquids in any one of the cation:anion ratios listed in Table 1.

### Formulation

In some embodiments, an ionic liquid provided herein is formulated in combination with a one or more drugs. In some embodiments, the ionic liquid can be combined with another solvent to enhance solubility and/or delivery. The solvent may be aqueous or non-aqueous. In some embodiments, the purpose of the solvent is to control the dose of the ionic liquid experienced by the mucus membrane or the gastrointestinal tract. Dilution of the ionic liquid by the solvent can serve the purpose of delivering a safe dose to the subject. In some embodiments, the purpose of the solvent is to improve solubility of the one or more drugs. Such improvements may come from the ability of the solvent to control the physicochemical environment of the ionic liquid to match the chemical properties of the one or more drugs. In some embodiments, the solvent may serve the purpose of improving the delivery across the mucosal membrane.

The solvents used may include without limitation: sterile water, saline solution, glycerin, propylene glycol, ethanol, oils, ethyl oleate, isopropyl myristate, benzyl benzoate, or surfactants.

In some embodiments, the solvent is chosen so as to not adversely impact the compatibility of the ionic liquid with the capsule.

In some embodiments, the one or more drugs may form micelles or other self-assembled structures. In some embodiments, such structures may occur only in the presence of ionic liquids.

In some embodiments, the one or more drugs is a nucleic acid molecule. A nucleic acid molecule, as described herein, can be a vector, an expression vector, an inhibitory nucleic acid, an aptamer, a template molecule or cassette (e.g., for gene editing), or a targeting molecule (e.g., for CRISPR-Cas technologies), or any other natural or synthetic nucleic acid molecule intended for delivery to an organism.

In any of the embodiments, the one or more drugs may be designed with the intent of treating a local tissue, e.g., the mucosal membrane of the intestine, treating a distant tissue, e.g., the liver, or entering systemic circulation.

In some embodiments, a composition as described herein, e.g., a composition comprising ionic liquids and one or more drugs, can further comprise a pharmaceutically acceptable excipient. Suitable excipients include, for example, water, saline, glycerol, ethanol, or the like, and combinations thereof. In addition, if desired, the composition can contain minor amounts of additional excipients such as emulsifying agents, surfactants, pH buffering agents, and the like, which enhance the effectiveness of the one or more drugs.

In some embodiments, the composition comprising an ionic liquid may be further encapsulated in a dosage form designed to facilitate delivery to an organism. Non-limiting examples of such dosage forms include capsules, tablets, and syrups.

In some embodiments, formulation may require excipients sugars (such as lactose), starches (such as corn starch), cellulose, cellulose derivatives (such as sodium carboxymethyl cellulose), gelatin, and other compatible substances.

In some embodiments, a composition comprising an ionic liquid described herein further comprises one or more additional agents. In some embodiments, the one or more additional agents are selected from a nucleic acid, a small molecule, and a polypeptide. In some embodiments, the one or more additional agents comprise a nucleic acid. In some embodiments, the one or more additional agents comprise a small molecule. In some embodiments, the one or more additional agents comprise a polypeptide. In some embodiments the polypeptide comprises an Antibody. In some embodiments, the Antibody comprises any one selected from Fragment Antigen-binding (Fab, F(ab′)₂), single chain variable fragment (scFv), and Nanobodies.

In some embodiments, the one or more additional agents are selected from a glucagon-like peptide (GLP-1), a glucagon-like peptide derivative, and a glucagon-like peptide mimetic. In some embodiments, the one or more additional agents are selected from liraglutide, exenatide, and semaglutide. In some embodiments, the one or more additional agents comprise liraglutide.

In some embodiments, the one or more additional agents are selected from insulin and pramlintide.

### Ionic Liquids for the Treatment of Diseases and Disorders

Provided herein, in some embodiments, is a method of treating a metabolic disease or disorder in a subject in need thereof, comprising administering a composition comprising an ionic liquid. Metabolic disorders include but are not limited to obesity, diabetes, fatty liver disease, or non-alcoholic fatty liver disease.

Provided herein, in some embodiments, is the use of ionic liquids for treating diabetes by oral administration. Oral administration can be achieved in any one of the dosing forms including pills, caplets, capsules, aerosol sprays, or liquids. The ionic liquid or the one or more drugs to be delivered with the ionic liquid can be encapsulated in a capsule. The ionic liquid with the dosing form may be present in any of the physical forms including a clear neat ionic liquid, a homogenous mixture of an ionic liquid with a pharmaceutically acceptable diluent, an emulsion, or a suspension. The oral dose can also be given as a syrup, a spray, or an aerosol. The composition of any oral dose disclosed herein may contain a predetermined amount of ionic liquid and optionally one or more drugs, and may be prepared by methods of pharmacy well known to those skilled in the art.

In some embodiments, described herein is a method of treatment of diabetes comprising orally administering an oral formulation of insulin in combination with an ionic liquid.

In some embodiments, described herein is a method of treatment of diabetes comprising orally administering an oral formulation of insulin and pramlintide in combination with an ionic liquid.

In some embodiments, described herein is a method of treatment of diabetes comprising orally administering an oral formulation of liraglutide or exenatide in an ionic liquid.

As described herein, ionic liquids are able to safely carry active compounds across the mucus membranes encountered during oral administration.

As described in the examples herein, when administered together with one or more drugs, ionic liquids solubilize the one or more drugs and result in enhanced delivery into systemic circulation. Accordingly, they are particularly suitable as delivery vehicles to and/or across mucus membranes.

In some embodiments, provided herein is a method of delivery of one or more drugs, the method comprising administering the one or more drugs in combination with an ionic liquid to a mucus membrane, e.g., a nasal, oral, or vaginal membrane.

In some embodiments, provided herein is a method of delivery of one or more drugs, the method comprising administering the one or more drugs at the dose of at least 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2500 mg/kg, 3000 mg/kg, 3500 mg/kg, 4000 mg/kg, 4500 mg/kg, 5000 mg/kg, 5500 mg/kg, 6000 mg/kg, 6500 mg/kg, 7000 mg/kg, 7500 mg/kg, 8000 mg/kg, 8500 mg/kg, 9000 mg/kg, or 10000 mg/kg. In some embodiments, provided herein is a method of delivery of one or more drugs, the method comprising administering the one or more drugs at the dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2500 mg/kg, 3000 mg/kg, 3500 mg/kg, 4000 mg/kg, 4500 mg/kg, 5000 mg/kg, 5500 mg/kg, 6000 mg/kg, 6500 mg/kg, 7000 mg/kg, 7500 mg/kg, 8000 mg/kg, 8500 mg/kg, 9000 mg/kg, or 10000 mg/kg.

### Glucagon-Like Peptide-1 (GLP-1)

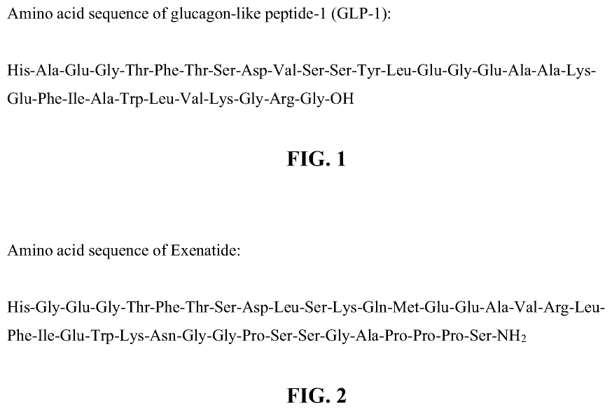

Glucagon-Like Peptide-1 (GLP-1) is a peptide hormone known to reduce food intake and hunger in humans. The amino acid sequence of GLP-1 is shown in FIG. 1. GLP-1 is an incretin derived from the transcription product of the proglucagon gene that contributes to glucose homeostasis. Because natural GLP-1 has an extremely short half-life, making its use as a therapeutic challenging, modified versions of GLP-1 with greater stability have been developed. Such modifications can be performed either by varying the sequence of the peptide or by conjugating another entity to the peptide. A common modification includes attachment of a lipid tail. GLP-1 mimetics are currently being used in the treatment of Type 2 diabetes, with recent clinical trials demonstrating that these treatments improve glucose homeostasis. They also help in achieving weight loss.

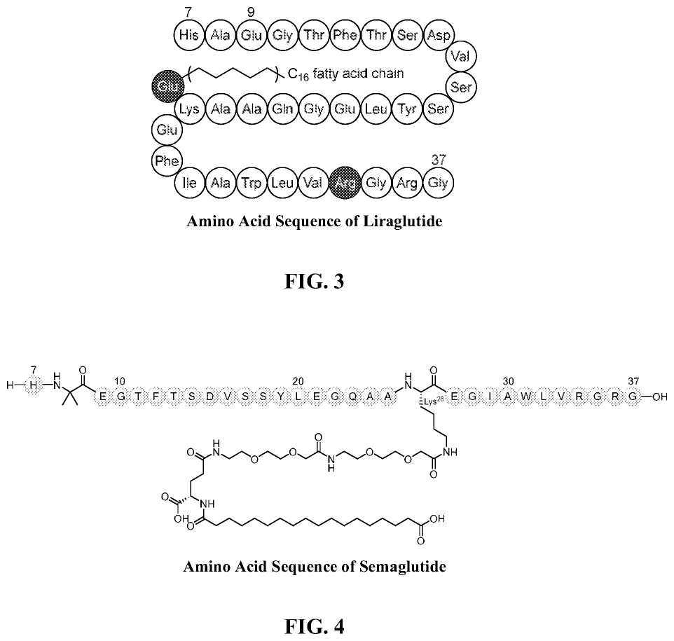

Various GLP-1 mimetics are known and used in the treatment of diabetes. GLP-1 mimetics (or analogs) can include exenatide. The amino acid sequence of Exenatide is shown in FIG. 2. Other examples of GLP-1 analogs include derivatives for reducing enzymatic degradation, e.g., lixisenatide, dulaglutide, semaglutide, albiglutide, liraglutide, and taspoglutide. The amino acid sequence of Liraglutide is shown in FIG. 3. The amino acid sequence of Semaglutide is shown in FIG. 4.

In some embodiments, described herein is a method of treatment of diabetes comprising orally administering an oral formulation of a GLP-1 polypeptide or mimetic/analog thereof in combination with ionic liquid.

In some embodiments, the compositions provided herein can be used to treat obesity by delivering a composition comprising an ionic liquid and a GLP-1 analog.

In some embodiments, the compositions provided herein can be used to treat obesity by dual action of the ionic liquid and the GLP-1 analog. Certain ionic liquids reduce fat absorption across the intestinal mucosa. The net result of the composition on reduced body weight may arise from a combination of reduced fat absorption, reduced food uptake, and increased delivery of a GLP-1 analog.

### EXAMPLES

### General

All animal experiments were performed in accordance with the animal care committee guidelines and the Guide for the Care and Use of Animals of the Institute of Laboratory Animal Resources, National Research Council.

### Example 1: Preparation of Choline Citronellate 1:2

To two equivalents of neat citronellic acid (20 g, 0.117 mol, 2 equiv.) in a 500 mL round bottom flask was added 12.13 g of an 80 wt % solution of choline bicarbonate (9.70 g, 0.059 mol, 1 equiv.). The mixture was stirred at 40° C. until CO₂ evolution ceased. Solvent was removed by rotary evaporation at 60° C. for 1 hour, and the product was dried in a vacuum oven for 48 hours at 60° C.

### Example 2: Preparation of Choline Cinnamate 1:2

To two equivalents of neat cinnamic acid (30 g, 0.202 mol, 2 equiv.) in a 500 mL round bottom flask was added 20.91 g of an 80 wt % solution of choline bicarbonate (16.72 g, 0.101 mol, 1 equiv.). Ethanol (5 mL) was added to mixture in order to solubilize the cinnamic acid. The mixture was stirred at 40° C. until CO₂ evolution ceased. Solvent was removed by rotary evaporation at 60° C. for 1 hour, and the product was dried in a vacuum oven for 48 hours at 60° C.

### Example 3: Preparation of Ionic Liquids

Several ionic liquids comprising choline as a cation and various anions were synthesized. To prepare ionic liquids, 2, 1, 0.5, or 0.33 equivalents of choline bicarbonate (80 wt % solution) were added to neat carboxylic acid anion in a 250-mL round bottom flask. For anions not miscible with the aqueous choline bicarbonate solution, a co-solvent, such as ethanol, was added until a homogenous mixture formed. The mixture was stirred at room temperature until CO₂ evolution ceased. Solvent was removed by rotary evaporation at 60° C. for 20 minutes, and each product was dried in a vacuum oven for 48 hours at 60° C.

Using 62 different anions, 108 different ionic liquids were synthesized. Of these 108 ionic liquids, 43 were solids at room temperature and 65 were liquids at room temperature. In some instances, ionic liquids that were non-flowable waxy solids or spreadable greasy solids liquified at elevated temperature (>30° C.). In some instances, solid powders did not liquefy at elevated temperature (>30° C.). In some instances (for example, decanoic acid), a single anion resulted in a liquid at one ratio (2:1) and in a solid at another ratio (1:1 or 1:2). The various ionic liquids formed and their physical characteristics are summarized in Table 1:

TABLE 1
Physical Properties of Ionic Liquids

| | | Physical Form |
|---|---|---|
| Anion | Cation | (Ratios Listed as Cation:Anion) |
| (R)-α-Lipoic Acid | Choline | Solid at 2:1, 1:1, 1:2, |
| 2-(4-Isobutylphenyl)propionic Acid | Choline | Solid at 1:1 |
| 2-(4,4-Dimethyl-2-pentanyl)-5,7,7- | Choline | Solid at 1:1 |
| trimethyloctanoic Acid | | |
| 2-Hexyldecanoic Acid | Choline | Liquid at 1:1 |
| 2-Hydroxyhippuric Acid | Choline | Solid at 1:1 |
| 3,7-Dimethyloctanoic Acid | Choline | Solid at 1:1, 1:2 |
| 4-Methylhexanoic Acid | Choline | Liquid at 1:1, 1:2 |
| 4-Methyloctanoic Acid | Choline | Liquid at 1:1, Solid at 1:2 |
| 4-Methylvaleric Acid | Choline | Liquid at 1:2 |
| 5-Norbornene-2-carboxylic Acid | Choline | Solid at 1:1 |
| Abietic Acid | Choline | Solid at 1:1 |
| Acetic Acid | Choline | Liquid at 2:1, 1:1, 1:2 |
| Arachidonic Acid | Choline | Solid at 1:1 |
| Caffeic Acid | Choline | Solid at 1:1 |
| Cinnamic Acid | Choline | Liquid at 1:1, 1:1.5, 1:2 |
| Citric Acid | Choline | Liquid at 2:1, 3:1, 4:1; Solid at 1:1 |
| Citronellic Acid | Choline | Liquid at 2:1, 1:1, 1:2 |
| Crotonic Acid | Choline | Liquid at 1:1, 1:2 |
| D-(+)-Galactonic Acid | Choline | Solid at 1:1 |
| Decanoic Acid | Choline | Liquid at 2:1; Solid at 1:1, 1:2 |
| Deoxycholic Acid | Choline | Solid at 2:1, 1:1 |
| Dihydrobenzoic Acid | Choline | Solid at 1:1 |
| Eicosapentanoic Acid (EPA) | Choline | Solid at 1:1 |
| Ellagic Acid | Choline | Solid at 1:1 |
| Fumaric Acid | Choline | Liquid at 2:1, Solid at 1:1 |
| Geranic Acid | Choline | Liquid at 1:1, 1:2 |
| Glutaric Acid | Choline | Liquid at 2:1, 1:1 |
| Glycolic Acid | Choline | Liquid at 2:1, 1:1 |
| Hexanoic Acid | Choline | Liquid at 1:1, 1:2 |
| Hydrocinnamic Acid (3-Phenylpropionic | Choline | Liquid at 1:1, 1:2 |
| Acid) | | |
| Isovaleric Acid | Choline | Liquid at 1:2 |
| L-(+)-Tartaric Acid | Choline | Liquid at 2:1; Solid at 1:1 |
| L-Ascorbic Acid | Choline | Solid at 1:1 |
| L-Glutathione reduced | Choline | Solid at 2:1 |
| Lactic Acid | Choline | Liquid at 1:1, 1:2 |
| Lauric Acid | Choline | Solid at 1:1, 1:2 |
| Levulinic Acid | Choline | Liquid at 1:1, 1:2 |
| Linoleic Acid | Choline | Liquid at 1:2; Solid at 1:1 |
| Linolenic Acid | Choline | Liquid at 1:2 |
| Maleic Acid | Choline | Liquid at 2:1, 1:1 |
| Malonic Acid | Choline | Liquid at 1:1 |
| Mesaconic Acid | Choline | Solid at 1:1; Liquid at 2:1 |
| Nonanoic Acid | Choline | Solid at 1:1, 1:2 |
| Octanoic Acid | Choline | Solid at 1:2, Liquid at 1:1 |
| Oleic Acid | Choline | Solid at 1:1, 1:2 |
| p-Toluenesulfonic Acid | Choline | Solid at 1:1; Liquid at 1:2 |
| Perillic Acid | Choline | Solid at 1:1 |
| Phosphoric Acid | Choline | Solid at 1:1; Liquid at 2:1, 1:2 |
| Pimelic Acid | Choline | Liquid at 2:1, 1:1 |
| Propionic Acid | Choline | Liquid at 2:1, 1:1 |
| Pyroglutamic Acid | Choline | Solid at 1:1; Liquid at 1:2 |
| Pyruvic Acid | Choline | Liquid at 1:1, 1:2 |
| Ricinoleic Acid | Choline | Liquid at 1:1, 1:2 |
| Sorbic Acid | Choline | Liquid at 1:1, 1:2 |
| Syringic Acid | Choline | Solid at 1:1 |
| Trans-2-Decenoic Acid | Choline | Solid at 1:1, 1:2 |
| Trans-2-Hexenoic Acid | Choline | Liquid at 1:1, 1:2 |
| Trans-2-Octenoic Acid | Choline | Liquid at 1:1, 1:2 |
| Trans-3-Octenoic Acid | Choline | Liquid at 1:1, 1:2 |
| Trans-7-Octenoic Acid | Choline | Liquid at 1:1, 1:2 |
| Trans-Ferulic Acid | Choline | Solid at 1:1 |
| Undecanoic Acid | Choline | Solid at 1:1 |
| Vanillic Acid | Choline | Solid at 1:1 |
| α-Ketoglutaric Acid | Choline | Liquid at 2:1 |
| Succinic Acid | Choline | Liquid at 2:1, 1:1, 1:2 |
| Malic Acid | Choline | Liquid at 2:1, 1:1 |
| Mandelic Acid | Choline | Liquid at 1:1, 1:2 |

### Example 4: Physical Form of Ionic Liquids and Deep Eutectic Solvents

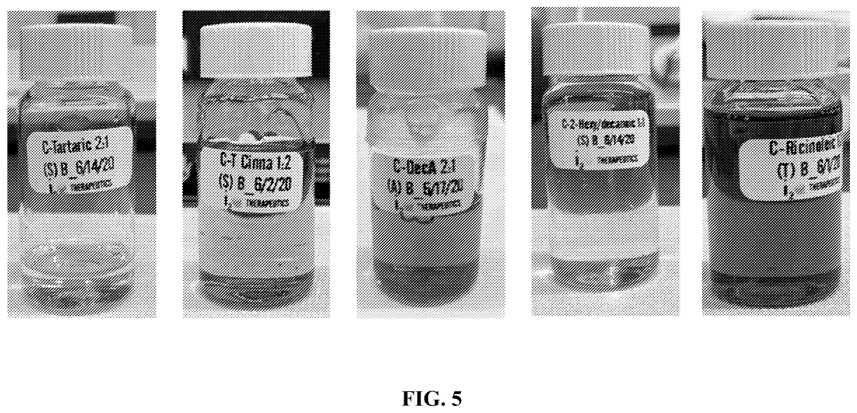

Ionic liquids varied significantly in their appearance and properties. Some ionic liquids, such as choline-tartaric acid (2:1) are clear liquids at room temperature. Others, such as choline-cinnamic acid are viscous yellow liquids at room temperature. Various ionic liquids which are liquid at room temperature are shown in FIG. 5.

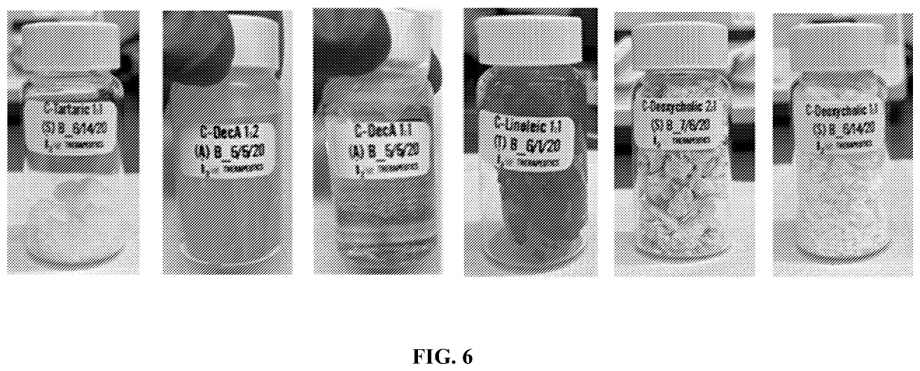

Some mixtures of anion and cation are not a liquid at room temperature. For example, choline-tartaric acid (1:1) is a solid, and choline-decanoic acid (1:1) is a waxy solid. Various non-liquid compositions are shown in FIG. 6. Ionic liquids that exist in a liquid form at room temperature are particularly suitable for the pharmaceutical applications described herein.

### Example 5: Effect on Blood Glucose Levels Following Choline-Citronellic Acid Administration

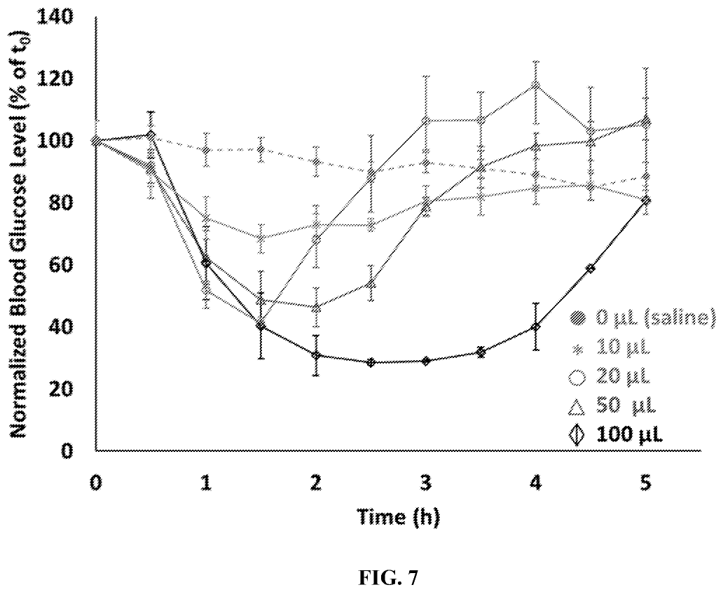

Adult non-diabetic male Wistar rats were fasted overnight but given free access to water and subsequently dosed with choline-citronellic acid via intrajejunal injection. As fasting control group was dosed with a saline solution via intrajejunal injection. About 250 μL of blood was collected at regular intervals in order to determine the blood glucose level. The obtained values, plotted as mean percent change±standard error in blood glucose levels with respect to initial reading (n=3) versus time, are shown in FIG. 7.

Choline-citronellic produced an immediate and dose-dependent decrease in blood glucose levels. Control treatment with saline did not change the blood glucose level from baseline. However, when the rats were administered 10, 20, 50 and 100 μL of choline-citronellic acid, the blood glucose level dropped in a dose-dependent manner. For a 100 μL dose, the glucose level dropped by about 70% from baseline. This scale of reduction in blood glucose level would likely be efficacious in the treatment of diabetes.

### Example 6: Effect on Blood Glucose Levels Following Choline-Octanoic Acid and Choline-Octenoic Acid Administration

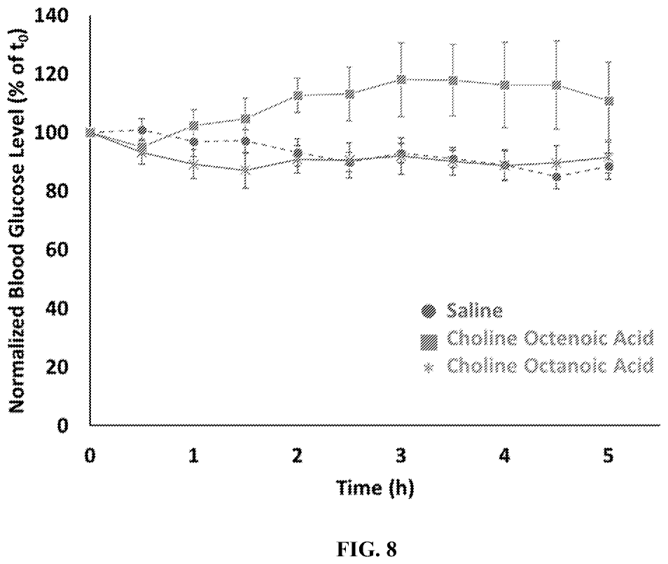

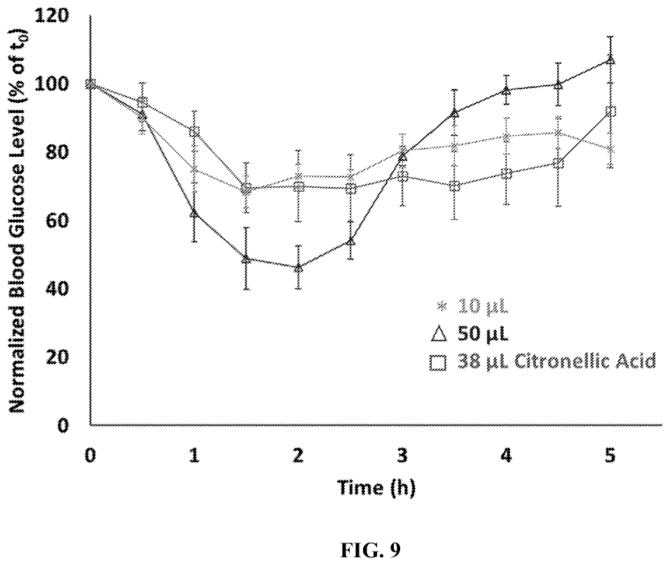

Adult non-diabetic male Wistar rats were fasted overnight but given free access to water and subsequently dosed with 100 μL choline-octanoic acid or choline-octenoic acid via intrajejunal injection. As fasting control group was dosed with a saline solution via intrajejunal injection. About 250 μL of blood was collected at regular intervals in order to determine the blood glucose level. The obtained values, plotted as mean percent change±standard error in blood glucose levels with respect to initial reading (n=3) versus time, are shown in FIG. 8.

Surprisingly, unlike choline-citronellic acid, which induced a pronounced decrease in blood glucose levels, neither choline-octanoic acid nor choline-octenoic acid decreased blood glucose levels in rats.

### Example 7: Effect on Blood Glucose Levels Following Citronellic Acid Administration

The obtained values, plotted as mean percent change±standard error in blood glucose levels with respect to initial reading (n=4) versus time, are shown in FIG. 9.

Adult non-diabetic male Wistar rats were fasted overnight but given free access to water and subsequently dosed with choline-citronellic acid or citronellic acid alone via intrajejunal injection. A 50 μL dose of choline-citronellic acid produced an immediate decrease in blood glucose levels to about 50% of starting levels. However, when the rats were administered 38 μL of citronellic acid alone, the equivalent of the acid content in 50 μL of the choline-citronellic acid ionic liquid, the blood glucose levels only dropped to about 30% of starting levels, a level similar to that observed following a 10 μL ionic liquid dose. Citronellic acid alone does not provide the same efficacy in lowering blood glucose as the choline-citronellic acid ionic liquid.

### Example 8: Reduction in Blood Glucose Levels Following Oral and Subcutaneous Choline-Citronellic Acid Administration

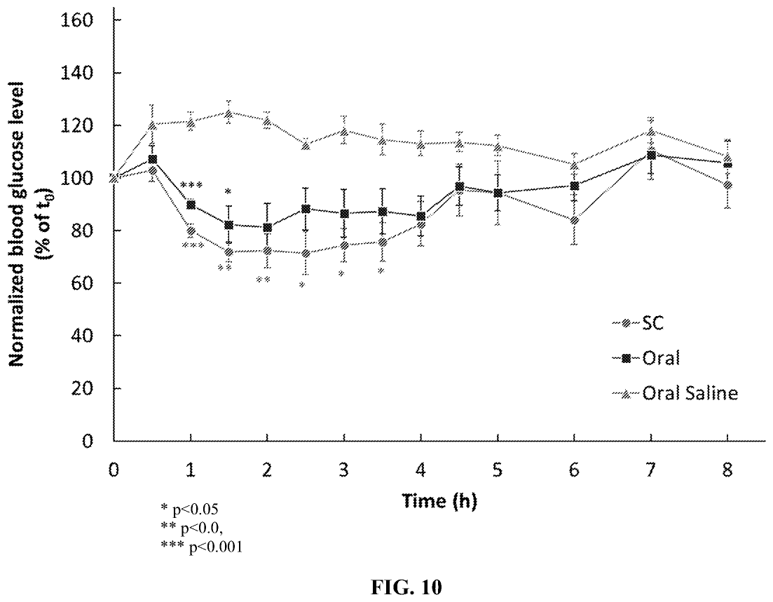

Adult non-diabetic male Wistar rats were fasted overnight but given free access to water and subsequently dosed with choline-citronellic acid via either subcutaneous injection or liquid gavage. A fasting control group was dosed orally with a saline solution. About 250 μL of blood was collected at regular intervals in order to determine the blood glucose level. The obtained values, plotted as mean percent change±standard error in blood glucose levels with respect to initial reading (n=4) versus time, are shown in FIG. 10.

Both oral and subcutaneous delivery of choline-citronellic acid resulted in reduced blood glucose levels in rats. In both cases, the glucose level dropped within the first two hours and increased over time.

### Example 9: Reduction in Blood Glucose Levels Following Oral Choline-Citronellic Acid Administration in a Rat Model of Type 1 Diabetes

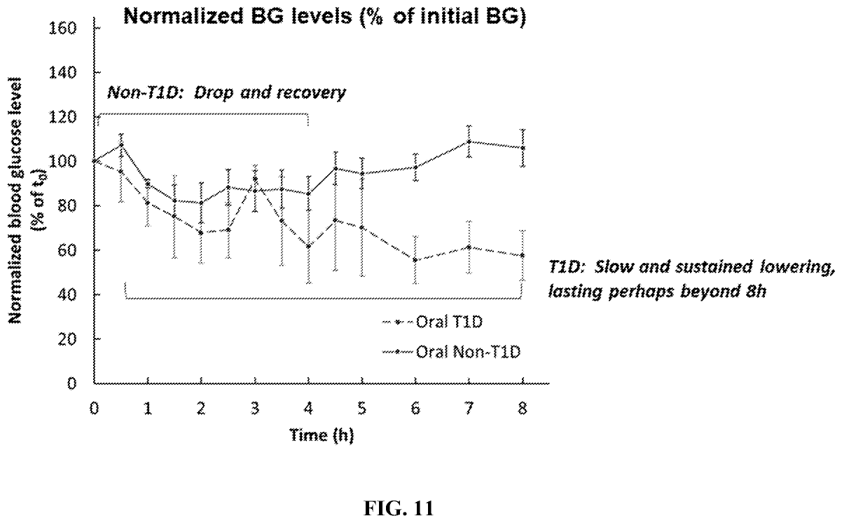

Adult Streptozocin-induced diabetic male Wistar rats and non-diabetic rats were fasted overnight but given free access to water and subsequently orally dosed with choline-citronellic acid via liquid gavage. About 250 μL of blood was collected at regular intervals in order to determine the blood glucose level. The obtained values, plotted as mean percent change±standard error in blood glucose levels with respect to initial reading (n=4) versus time, are shown in FIG. 11.

Oral delivery of choline-citronellic acid produced a substantial drop in blood glucose levels. In a healthy rat, the glucose level dropped and recovered slowly with time. In the Type 1 diabetes model, on the other hand, the glucose level continued to drop after oral administration of choline-citronellic acid, demonstrating the potential of choline-citronellic acid as a therapeutic for Type 1 diabetes.

### Example 10: Induced Insulation Secretion Following Choline-Citronellic Acid Administration in a Rat Model of Type 1 Diabetes

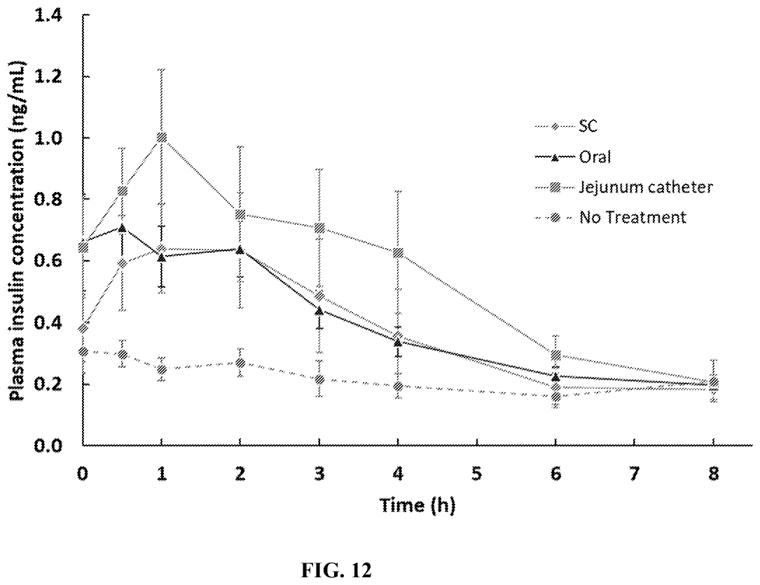

Adult Streptozotocin-induced diabetic male Wistar rats were fasted overnight but given free access to water and subsequently dosed with choline-citronellic acid via subcutaneous injection (SC), oral liquid gavage, or jejunum placement via a catheter (Jejunum catheter). A fasting control group was not treated. About 250 μL of blood was collected at regular intervals in order to determine the insulin serum concentration. The obtained values, plotted as mean plasma insulin concentration±standard error (n=3) versus time, are shown in FIG. 12.

Stimulation of insulin secretion was observed for all administration methods of choline-citronellic acid, demonstrating the potential of choline-citronellic acid as a therapeutic for Type 1 diabetes patients who lack the natural ability to produce insulin in the pancreas. Choline-citronellic acid could be particularly useful for newly diagnosed Type 1 diabetes patients or prediabetic patients, for whom the pancreas still maintains some insulin-producing cells which can be stimulated by choline-citronellic acid administration to produce insulin.

### Example 11: Increased Glucose Excretion in Urine Following Choline-Citronellic Acid Administration

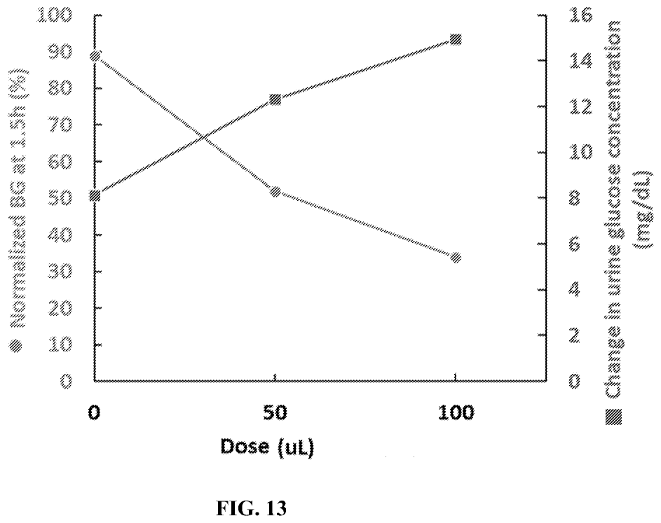

Adult non-diabetic male Wistar rats were fasted overnight but given free access to water and subsequently dosed via intrajejunal injection with choline-citronellic acid at varying doses and monitored for blood glucose. At 1.5 hours post-injection, urine was sampled from the bladder to determine glucose concentration. The obtained glucose concentration values, plotted as percent change in blood glucose levels with respect to initial reading versus dose level and change in glucose concentration in the urine collected from the bladder versus dose level (n=1), are shown in FIG. 13.

As evidenced by the declining blood glucose levels and increasing glucose concentration in the urine, choline-citronellic acid reduced the ability of the kidneys to reabsorb glucose, enhancing the amount of glucose removal from the body and helping to reduce blood glucose levels.

### Example 12: Delivery of Liraglutide With Choline-Hydrocinnamic Acid

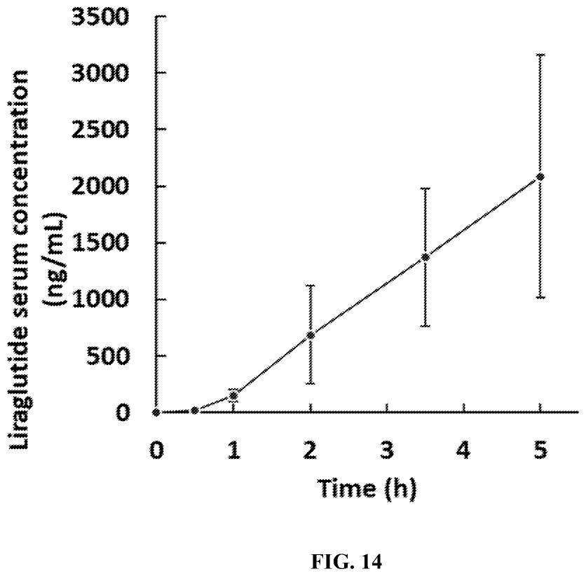

Adult non-diabetic male Wistar rats were fasted overnight but given free access to water and subsequently dosed via intrajejunal injection with liraglutide and choline-hydrocinnamic acid. A fasting control group was dosed with a saline solution via intrajejunal injection. About 250 L of blood was collected at hourly intervals in order to determine the liraglutide serum concentration. The obtained values, plotted as mean liraglutide serum concentration±standard error (n=3) in ng/mL versus time, are shown in FIG. 14.

When delivered as a choline-hydrocinnamic acid formulation, liraglutide was delivered into blood circulation with surprisingly high serum concentration. Oral delivery of liraglutide is known to be difficult. For example, Buckley and coworkers demonstrated only minute absorption of liraglutide even in the presence of well-known permeation enhancers (Sci. Transl. Med. 2018, 10, eaar7047). The blood concentrations of liraglutide reported herein are approximately 4,400-fold greater than those reported in the literature. This unexpected level of liraglutide delivery demonstrates the promise of choline-hydrocinnamic acid as a diabetes therapeutic, especially for the treatment of Type 2 diabetes, for which the therapeutic benefits of liraglutide are well established. However, the fact that the current standard of care for liraglutide is daily injections poses a significant hurdle in compliance and patient acceptance. An oral pill that can deliver liraglutide would dramatically improve patient impact.

### Example 13: Delivery of Liraglutide with Various Choline-Based Ionic Liquids

Adult non-diabetic male Wistar rats were fasted overnight but given free access to water and subsequently dosed via intrajejunal injection with liraglutide and one of various choline-based ionic liquids. The peak 5-hour liraglutide concentrations obtained for the various ionic liquids are summarized in Table 2:

TABLE 2
Peak 5-Hour Concentration of Liraglutide Delivered Via Intrajejunal
Injection with Various Choline-Based Ionic Liquids

| | Peak 5-Hour Liraglutide |
|---|---|
| Ionic Liquid | Concentration (ng/mL) |
| Choline-Hydrocinnamic acid | 2086 |
| Choline-Cinnamic acid | 2057 |
| Choline-Glutaric acid | 365 |
| Choline-Malonic acid | 234 |
| Choline-Octenoic acid | 201 |
| Choline-Linoleic acid | 44 |
| Choline-Citronellic acid | 4.5 |

The amount of liraglutide delivered depended on the composition of the ionic liquid. Choline-citronellic acid yielded a modest but significant absorption to yield a peak concentration of 4.5 ng/mL. Choline-linoleic acid improved the concentration by about 10-fold to 44 ng/mL. Choline-malonic acid further improved the absorption to yield a blood liraglutide concentration of 365 ng/mL. Unexpectedly, choline-hydrocinnamic acid yielded a blood liraglutide concentration of greater than 2000 ng/mL, a 500-fold improvement over choline-citronellic acid.

### Example 14: Lira-C-Cinnamic 1:1/dogs—Delivery of Liraglutide with Choline-Cinnamic Acid 1:1 to the Stomach or Duodenum

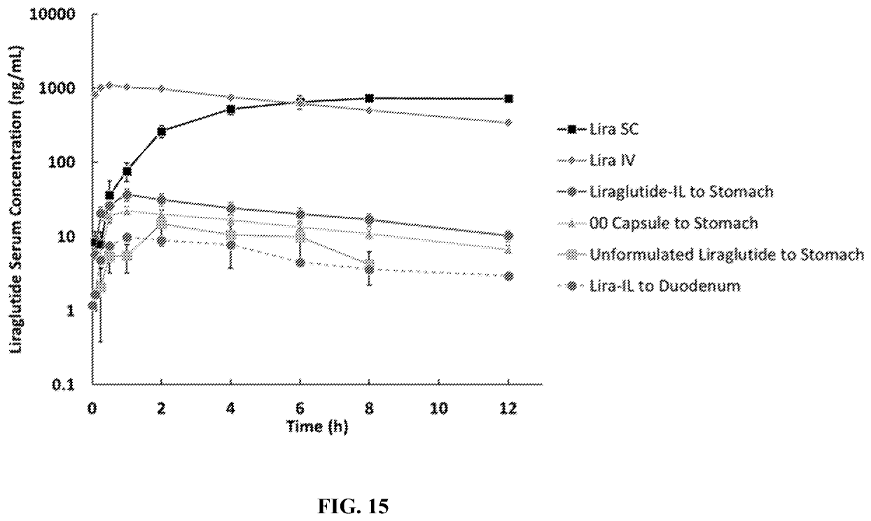

Adult non-diabetic male Beagle dogs were fasted overnight but given free access to water and subsequently dosed under anesthesia via endoscopic placement to the stomach (as a liquid or capsule) or duodenum (as a liquid) with 0.6 mg/kg Liraglutide with Choline-Cinnamic Acid 1:1. Dogs were recovered and plasma collected over a 12 h period. Control groups included intravenous (IV, 0.03 mg/kg) and subcutaneous (SC, 0.06 mg/kg) dosing. 0.6 mg/kg unformulated Liraglutide was also administered to the stomach endoscopically (FIG. 15).

### Example 15: Exenatide-C-Cinnamic 1:1/dogs—Delivery of Exenatide with Choline-Cinnamic Acid 1:1 to the Stomach

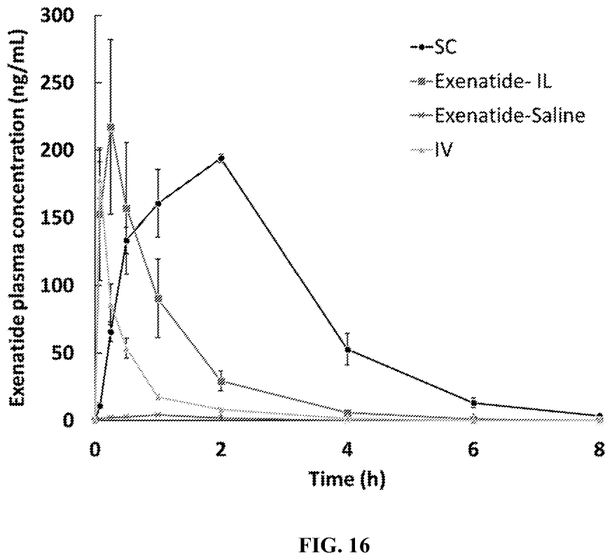

Adult non-diabetic male Beagle dogs were fasted overnight but given free access to water and subsequently dosed under anesthesia via endoscopic placement to the stomach with 0.6 mg/kg Exenatide with Choline-Cinnamic Acid 1:1 in liquid form (Exenatide-IL). Dogs were recovered and plasma collected over a 12 h period. Control groups included intravenous (IV, 0.03 mg/kg) and subcutaneous (SC, 0.06 mg/kg) dosing. Unformulated exenatide was also administered as a liquid in buffer (saline, 0.6 mg/kg) to the stomach endoscopically (FIG. 16).

### Example 16: Semaglutide-C-Cinnamic 1:1 Capsules/Dogs—Delivery of Semaglutide with Choline-Cinnamic Acid 1:1 to the Stomach in Gelatin and HPMC Capsules

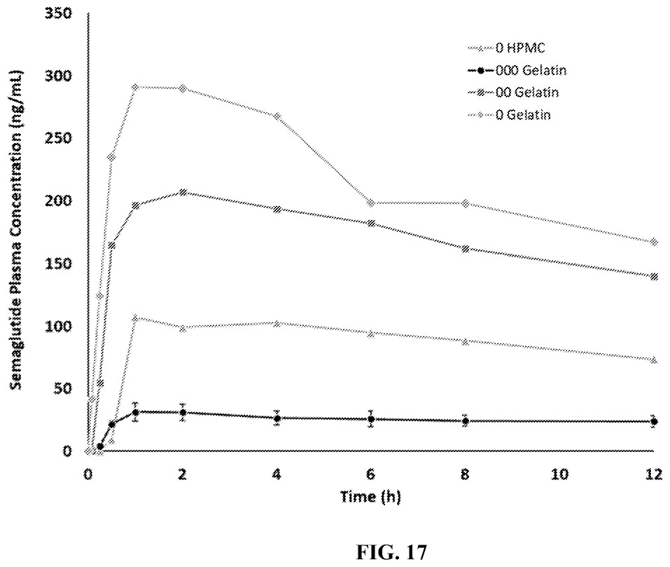

Adult non-diabetic male Beagle dogs were fasted overnight but given free access to water and subsequently dosed via endoscopic placement under anesthesia to the stomach 0.6 mg/kg Semaglutide with Choline-Cinnamic Acid 1:1 contained in 0, 00 or 000 gelatin capsules coated with Evonik's EPO coating or 0 HPMC capsule. Dogs were recovered and plasma collected over a 12 h period (FIG. 17).

### Example 17: Semaglutide-C-Cinnamic 1:1/Dogs—Delivery of Semaglutide with Choline-Cinnamic Acid 1:1 to the Stomach

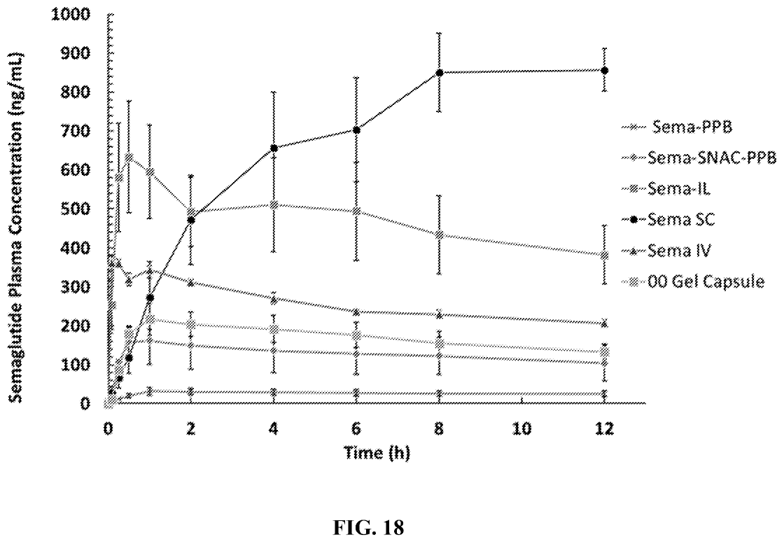

Adult non-diabetic male Beagle dogs were fasted overnight but given free access to water and subsequently dosed under anesthesia via endoscopic placement to the stomach with 0.6 mg/kg Semaglutide with Choline-Cinnamic Acid 1:1, either in liquid form (Sema-IL), or in a gelatin capsule (00 Gel Capsule). Dogs were recovered and plasma collected over a 12 h period. Control groups included intravenous (IV, 0.03 mg/kg) and subcutaneous (SC, 0.06 mg/kg) dosing. Unformulated semaglutide was also administered as a liquid in buffer (PPB, 0.6 mg/kg) to the stomach endoscopically. As a comparator, 0.6 mg/kg semaglutide was mixed with SNAC, a permeation enhancer and dosed via liquid to the stomach endoscopically (FIG. 18).

### Example 18: Mixture of Liraglutide/Exenatide—Co-Delivery of Liraglutide and Exenatide with Choline-Cinnamic Acid 1:1

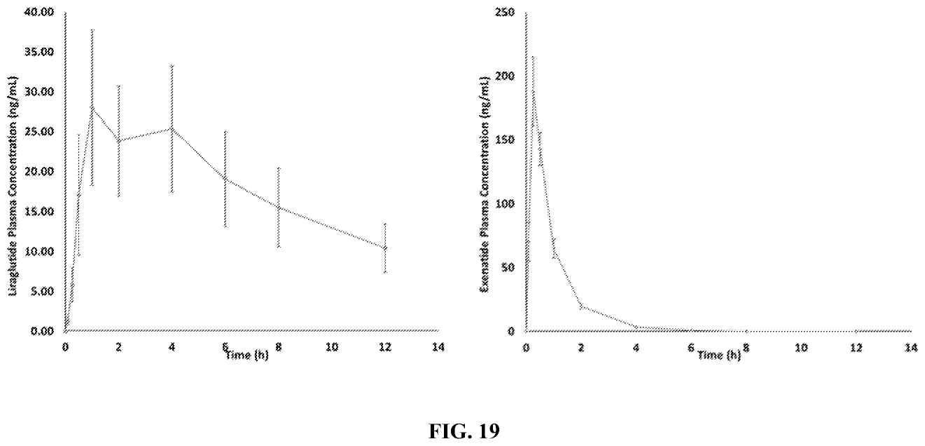

Adult non-diabetic male Beagle dogs were fasted overnight but given free access to water and subsequently dosed under anesthesia via endoscopic placement to the stomach with a mixture of 0.3 mg/kg each Liraglutide and Exenatide with Choline-Cinnamic Acid 1:1 in liquid form. Dogs were recovered and plasma collected over a 12 h period. (FIG. 19).

### Example 19: Safety/Tox—Safety Profile of Choline-Cinnamic Acid 1:1.5

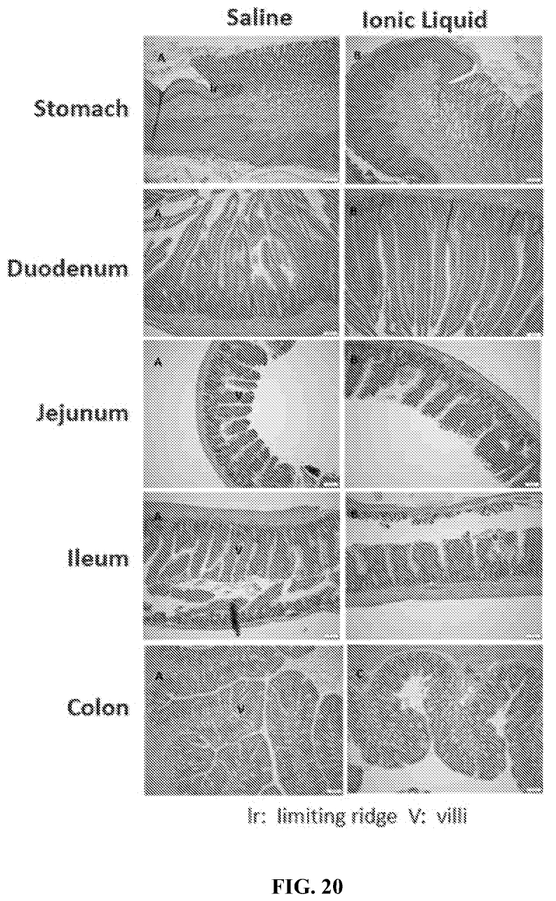

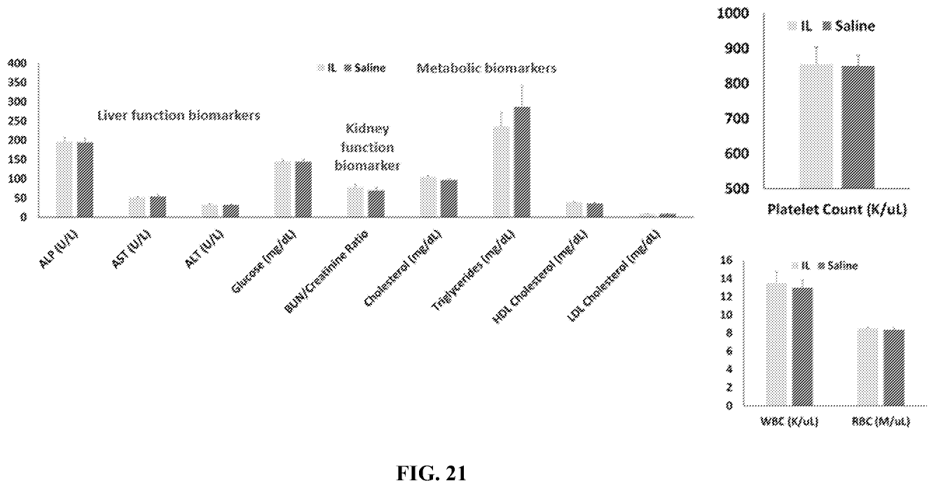

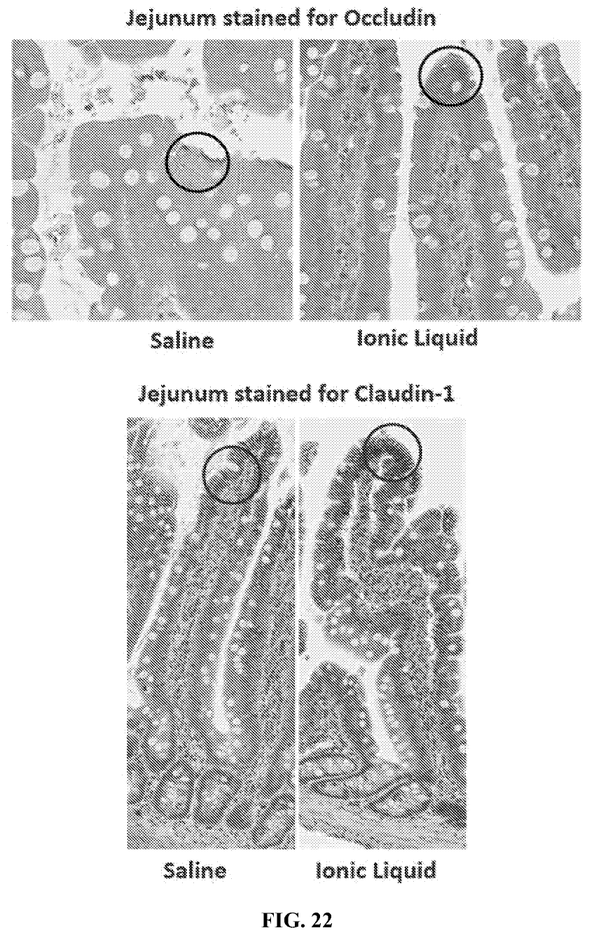

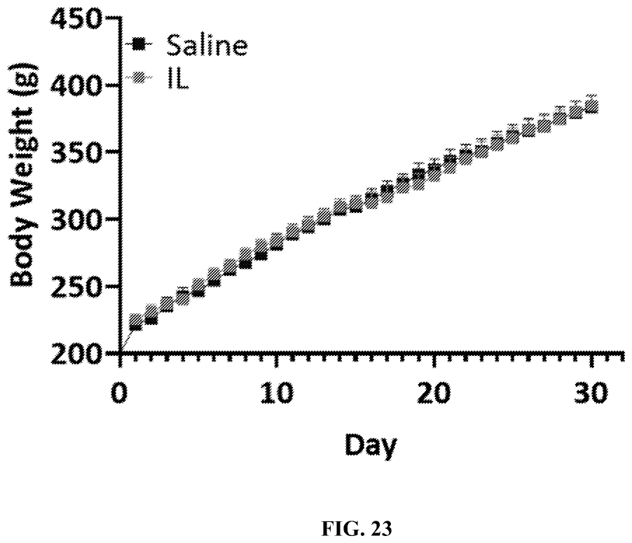

Adult non-diabetic male Wistar rats were dosed daily for 30 days via oral gavage with either 25 or 100 uL Choline-Cinnamic Acid 1:1.5. Two placebo groups received either 25 or 100 uL saline. Animals were observed daily for general health and weight recorded. On day 31, all animals were euthanized, and tissue, blood and plasma samples collected for analysis. Sections of organs (heart, lungs, liver, kidneys, and spleen) and all GI tract sections (stomach, duodenum, jejunum, ileum and colon) were stained with H&E and an expert pathologist read the results, concluding that there was no significant difference found in their histological examination of IL or saline (placebo) dosed animals' organs and GI tract tissues (FIG. 20). Blood cell counts were not statistically significantly different between the IL-dosed and placebo groups for either dose level, nor were plasma indicators of organ toxicity (FIG. 21). IHC staining of tight junction proteins Claudin-1 and Occludin of the GI tract tissues (duodenum, jejunum, ileum and colon) showed no difference between the IL-dosed and placebo groups for either dose level (FIG. 22). There was no difference in overall health or body weight gain between the IL-dosed and placebo groups (FIG. 23).

### Example 20: Delivery of Drugs Formulated with Choline-Cinnamic Acid in Various Cation:Anion Ratios to the Stomach or Duodenum

Adult non-diabetic male Beagle dogs are fasted overnight but given free access to water and subsequently dosed under anesthesia via endoscopic placement to the stomach (as a liquid or capsule (e.g., gelatin capsule)) or duodenum (as a liquid) with a drug, for example, Liraglutide, Exenatide, or Semaglutide, formulated with Choline-Cinnamic Acid in a ratio of, for example, from about 4:1 to about 1:4. For instance, Choline-Cinnamic Acid is formulated in any one of the ratio as described in paragraphs or [0087]. Dogs are recovered and plasma is collected over a 12 h period. Control groups include intravenous (IV) and subcutaneous (SC) dosing. The same dose of the unformulated drug, for example, Liraglutide, Exenatide, or Semaglutide, is also administered to the stomach endoscopically.

### Example 21: Delivery of Drugs Formulated with Various Ionic Liquids to the Stomach or Duodenum

Adult non-diabetic male Beagle dogs are fasted overnight but given free access to water and subsequently dosed under anesthesia via endoscopic placement to the stomach (as a liquid or capsule (e.g., gelatin capsule)) or duodenum (as a liquid) with a drug, for example, Liraglutide, Exenatide, or Semaglutide, formulated with Choline-Hydrocinnamic acid, Choline-Glutaric acid, Choline-Malonic acid, Choline-Octenoic acid, or Choline-Citronellic acid in a ratio of, for example, from about 4:1 to about 1:4. For instance, Choline-Hydrocinnamic acid, Choline-Glutaric acid, Choline-Malonic acid, Choline-Octenoic acid, or Choline-Citronellic acid is formulated at any of the ratio as described in paragraphs or [0087]. Dogs are recovered and plasma is collected over a 12 h period. Control groups include intravenous (IV) and subcutaneous (SC) dosing. The same dose of the unformulated drug, for example,. Liraglutide, Exenatide, or Semaglutide, is also administered to the stomach endoscopically.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1.-59. (canceled)

60. A composition comprising one or more agents, and an ionic liquid,
wherein the one or more agents are selected from the group consisting of a nucleic acid, a small molecule, and a polypeptide, and
wherein:
(A) (i) the ionic liquid is represented by Formula (I): 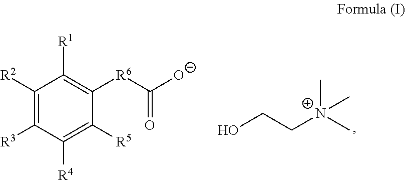
wherein:
R¹, R², R³, R⁴, and R⁵ are independently selected from hydrogen, halo, cyano, nitro, amino, C₁₋₆alkoxy, C₁₋₆heteroalkyl, C₁₋₆haloalkyl, C₁₋₆alkyl, C₂₋₆alkenyl, and C₂₋₆alkynyl; and
R⁶ is selected from C₁₋₆alkyl, C₂₋₆alkenyl, and C₂₋₆alkynyl;
(ii) the ionic liquid is represented by Formula (II): 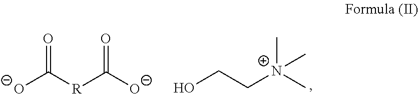
wherein:
R is selected from Ci-6alkyl, C2-6alkenyl, and C2-6alkynyl; or
(iii) the ionic liquid is represented by Formula (III): 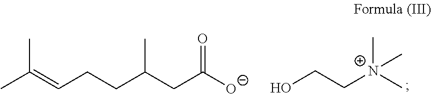
or
(B) the ionic liquid comprises an anionic component selected from the group consisting of (R)-α-lipoic acid, 2-(4-isobutylphenyl)propionic acid, 2-(4,4-dimethyl-2-pentanyl)-5,7,7-trimethyloctanoic acid, 2-hexyldecanoic acid, 2-hydroxyhippuric acid, 3,7-dimethyloctanoic acid, 4-methylhexanoic acid, 4-methyloctanoic acid, 4-methylvaleric acid, 5-norbornene-2-carboxylic acid, abietic acid, acetic acid, arachidonic acid, caffeic acid, cinnamic acid, citric acid, citronellic acid, crotonic acid, D-(+)-galactonic acid, decanoic acid, deoxycholic acid, dihydrobenzoic acid, eicosapentanoic acid, ellagic acid, fumaric acid, geranic acid, glutaric acid, glycolic acid, hexanoic acid, hydrocinnamic acid (3-phenylpropionic acid), isovaleric acid, L-(+)-tartaric acid, L-ascorbic acid, L-glutathione reduced, lactic acid, lauric acid, levulinic acid, linoleic acid, linolenic acid, maleic acid, malonic acid, mesaconic acid, nonanoic acid, octanoic acid, oleic acid, p-toluenesulfonic acid, perillic acid, phosphoric acid, pimelic acid, propionic acid, pyroglutamic acid, pyruvic acid, ricinoleic acid, sorbic acid, syringic acid, trans-2-decenoic acid, trans-2-hexenoic acid, trans-2-octenoic acid, trans-3-octenoic acid, trans-7-octenoic acid, trans-ferulic acid, undecanoic acid, vanillic acid, α-ketoglutaric acid, succinic acid, malic acid, mandelic acid, and octenoic acid.

61. The composition of claim 60, wherein the one or more agents are selected from the group consisting of a glucagon-like peptide (GLP-1) or derivative or mimetic thereof, insulin, and pramlintide.

62. The composition of claim 60, wherein the one or more agents are liraglutide, exenatide, semaglutide, or any combination thereof.

63. The composition of claim 60, wherein the composition comprises semaglutide, and choline-cinnamic acid.

64. The composition of claim 60, wherein the ionic liquid is represented by Formula (I), and wherein:
(i) at least two of R¹, R², R³, R⁴, and R⁵ are hydrogen;
(ii) at least three of R¹, R², R³, R⁴, and R⁵ are hydrogen; or
(iii) R¹, R², R³, R⁴, and R⁵ are hydrogen.

65. The composition of claim 60, wherein the ionic liquid is represented by Formula (I), and wherein R⁶ is selected from the group consisting of C₁₋₆alkyl and C₂₋₆alkenyl.

66. The composition of claim 60, wherein the ionic liquid is represented by Formula (I), and wherein R⁶ is C₁₋₆alkyl, C₂alkyl, C₁₋₆alkenyl, or C₂alkenyl.

67. The composition of claim 60, wherein the ionic liquid is represented by Formula (II), and wherein R is C₁₋₆alkyl, C₁alkyl, or C₃alkyl.

68. The composition of claim 60, wherein the ionic liquid comprises a cholinium cation.

69. The composition of claim 60, wherein the ionic liquid comprises a cholinium cation and an anion selected from the group consisting of cinnamate, hydrocinnamate, malonate, citronellate, octenoate, linoleate, and glutarate.

70. The composition of claim 60, wherein the ionic liquid comprises choline-hydrocinnamic acid, choline-cinnamic acid, choline-glutaric acid, choline-malonic acid, choline-octenoic acid, choline-linoleic acid, or choline-citronellic acid.

71. The composition of claim 60, wherein:
(i) the composition comprises the ionic liquid at a concentration of at least 0.1% weight per volume;
(ii) the composition comprises the ionic liquid at a concentration of at least 0.05 M;
(iii) the ionic liquid comprises a cation:anion molar ratio of from about 4:1 to about 1:4; or
(iv) any combination thereof.

72. The composition of claim 60, wherein the ionic liquid comprises:
(i) choline and hydrocinnamic acid in a 1:2 molar ratio;
(ii) choline and cinnamic acid in a 1:1 molar ratio;
(iii) choline and glutaric acid in a 1:1 molar ratio;
(iv) choline and malonic acid in a 1:1 molar ratio;
(v) choline and octenoic acid in a 1:1 molar ratio;
(vi) choline and octenoic acid in a 1:2 molar ratio;
(vii) choline and citronellic acid in a 1:1 molar ratio; or
(viii) choline and citronellic acid in a 1:2 molar ratio.

73. The composition of claim 60, wherein the composition is formulated as a liquid-filled capsule, pills, caplets, aerosol sprays, or liquids.

74. The composition of claim 60, wherein the composition further comprises a pharmaceutically acceptable excipient.

75. A method of treating a disease or disorder in a subject in need thereof comprising administering to the subject a therapeutically effective amount of the composition of claim 60,
wherein the administering is effective to treat the disease or disorder in the subject.

76. The method of claim 75, wherein the disease or disorder is diabetes, or non-alcoholic steatohepatitis.

77. The method of claim 76, wherein the disease or disorder is diabetes, and the diabetes is Type 1 diabetes or Type 2 diabetes.

78. The method of claim 75, wherein:
(i) the composition is administered via subcutaneous, intravenous, or oral administration, or the composition is administered to a mucus membrane;
(ii) the composition is administered in a single dose or in multiple doses; or
(iii) any combination thereof.

79. A method of treating obesity, preventing weight gain, or reducing weight in a subject in need thereof comprising administering to the subject an effective amount of the composition of claim 60,
wherein the administering is effective to treat obesity, prevent weight gain, or reduce weight in the subject.

80. The method of claim 79, wherein:
(i) the composition is administered via subcutaneous, intravenous, or oral administration, or the composition is administered to a mucus membrane;
(ii) the composition is administered in a single dose or in multiple doses; or
(iii) any combination thereof.

81. A method of delivering one or more agents to a subject in need thereof comprising administering the one or more agents in combination with the composition of claim 60,
wherein the one or more agents are selected from the group consisting of a nucleic acid, a small molecule, and a polypeptide; and
wherein the delivering is more efficient relative to a method of delivering one or more agents without the composition.

82. The method of claim 81, wherein the one or more agents are selected from the group consisting of a glucagon-like peptide (GLP-1) or derivative or mimetic thereof, insulin, and pramlintide.

83. The method of claim 81, wherein the one or more agents are liraglutide, exenatide, semaglutide, or any combination thereof.

84. A composition comprising an ionic liquid, wherein:
(A) (i) the ionic liquid is represented by Formula (I): 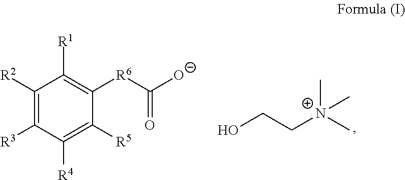
wherein:
R¹, R², R³, R⁴, and R⁵ are independently selected from hydrogen, halo, cyano, nitro, amino, C₁₋₆alkoxy, C₁₋₆heteroalkyl, C₁₋₆haloalkyl, C₁₋₆alkyl, C₂₋₆alkenyl, and C₂₋₆alkynyl; and
R⁶ is selected from C₁₋₆alkyl, C₂₋₆alkenyl, and C₂₋₆alkynyl;
(ii) the ionic liquid is represented by Formula (II): 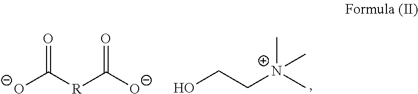
wherein:
R is selected from C₁₋₆alkyl, C₂₋₆alkenyl, and C₂₋₆alkynyl; or
(iii) the ionic liquid is represented by Formula (III): 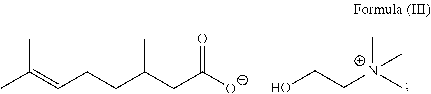
or
(B) the ionic liquid comprises an anionic component selected from the group consisting of (R)-α-lipoic acid, 2-(4-isobutylphenyl)propionic acid, 2-(4,4-dimethyl-2-pentanyl)-5,7,7-trimethyloctanoic acid, 2-hexyldecanoic acid, 2-hydroxyhippuric acid, 3,7-dimethyloctanoic acid, 4-methylhexanoic acid, 4-methyloctanoic acid, 4-methylvaleric acid, 5-norbornene-2-carboxylic acid, abietic acid, acetic acid, arachidonic acid, caffeic acid, cinnamic acid, citric acid, citronellic acid, crotonic acid, D-(+)-galactonic acid, decanoic acid, deoxycholic acid, dihydrobenzoic acid, eicosapentanoic acid, ellagic acid, fumaric acid, geranic acid, glutaric acid, glycolic acid, hexanoic acid, hydrocinnamic acid (3-phenylpropionic acid), isovaleric acid, L-(+)-tartaric acid, L-ascorbic acid, L-glutathione reduced, lactic acid, lauric acid, levulinic acid, linoleic acid, linolenic acid, maleic acid, malonic acid, mesaconic acid, nonanoic acid, octanoic acid, oleic acid, p-toluenesulfonic acid, perillic acid, phosphoric acid, pimelic acid, propionic acid, pyroglutamic acid, pyruvic acid, ricinoleic acid, sorbic acid, syringic acid, trans-2-decenoic acid, trans-2-hexenoic acid, trans-2-octenoic acid, trans-3-octenoic acid, trans-7-octenoic acid, trans-ferulic acid, undecanoic acid, vanillic acid, α-ketoglutaric acid, succinic acid, malic acid, mandelic acid, and octenoic acid.


SEQUENCE LISTING (4 sequences)

SEQ ID NO: 1 - protein, 31 aa, Homo sapiens
HAEGTFTSDVSSYLEGEAAKEFIAWLVKGRG

SEQ ID NO: 2 - protein, 37 aa, synthetic construct
  SITE 37: note = Amidated serine
HGEGTFTSDLSKQMEEAVRLFIEWKNGGPSSGAPPPS

SEQ ID NO: 3 - protein, 31 aa, synthetic construct
  SITE 20: note = Lysine side chain is linked via an amide linkage to Glu-C16 fatty acid chain
HAEGTFTSDVSSYLEGQAAKEFIAWLVRGRG

SEQ ID NO: 4 - protein, 31 aa, synthetic construct
  MOD_RES 2: note = Aib
  SITE 20: note = Lysine side chain is linked via an amide linkage to AEEAc-AEEAc-gamma-Glu-carboxyheptadecanoyl
HXEGTFTSDVSSYLEGQAAKEGIAWLVRGRG